(19) United States
(12) Patent Application Publication
(10) Pub. No.: US 2026/0272531 A1
(43) Pub. Date: Sep. 17, 2026

(54) PULSE STIMULATOR FOR CELL ABLATION AND METHOD THEREOF

(71) Applicant: National Taiwan University of Science and Technology, TAIPEI CITY (TW)

(72) Inventors: Fu-Ming WANG, New Taipei City (TW); Zong-Yu YANG, New Taipei City (TW); Peng-Hui WANG, Taipei City (TW)

(21) Appl. No.: 19/251,854

(22) Filed: Jun. 27, 2025

(30) Foreign Application Priority Data
Mar. 17, 2025 (TW) 114109908

(51) Int. Cl. A61B 18/00

(52) U.S. Cl. CPC A61B 18/00; A61B 2018/00577; A61B 2018/00708; A61B 2018/00714; A61B 2018/0072; A61B 2018/00732; A61B 2018/00761; A61B 2018/00767

(57) ABSTRACT

Provided is a pulse stimulator for cell ablation, including: a generator configured to deliver an electrical pulse to at least one electrode, the electrical pulse includes: a positive voltage, a negative voltage, or a combination thereof based on a resting membrane potential, or, a positive power, a negative power, or a combination thereof based on an extracellular membrane potential being 0 volt, in which the electrical pulse delivery frequency ranges from 0.001 Hz to less than 1 Hz to achieve non-thermal ablation of a plurality of cells in a target area.

### CROSS-REFERENCE TO RELATED APPLICATION

This application claims priority to Taiwan Application Serial Number 114109908, filed on Mar. 17, 2025, which is herein incorporated by reference in its entirety.

### BACKGROUND

### Field of Invention

The present invention relates to a pulse stimulator and method thereof. More particularly, the present invention relates to the pulse stimulator for cell ablation and method thereof.

### Description of Related Art

Ablation techniques include irreversible electroporation (IRE) and reversible electroporation. Irreversible electroporation is a method that uses high-energy pulsed electric fields to directly induce permanent and irreversible pore formation in the cell membrane, leading to cell death. If the potential is below a certain threshold, the poration phenomenon formed in the lipid bilayer of the cell membrane is reversible. The electrical pulse time mentioned above is generally limited to the range of nanoseconds to milliseconds. Compared with thermal ablation, irreversible electroporation does not rely on other heating or cooling devices, so the thermal effect on tissue is less significant.

### SUMMARY

The present disclosure provides a pulse stimulator for cell ablation, comprising: a generator configured to deliver an electrical pulse to at least one electrode. The electrical pulse comprises a positive voltage, a negative voltage, or a combination thereof based on a resting membrane potential; or a positive power, a negative power, or a combination thereof based on an extracellular membrane potential being 0 volt (V). Wherein a delivery frequency of the electrical pulse comprises from 0.001 hertz (Hz) to less than 1 Hz, and wherein the electrical pulse is configured to non-thermal ablate a plurality of cells in a target area.

In some embodiments, the positive power based on the extracellular membrane potential comprises from greater than 0 microwatt (μW) to 100 microwatts, and the negative power based on the extracellular membrane potential comprises from less than 0 microwatt to −100 microwatts.

In some embodiments, the positive voltage based on the resting membrane potential comprises from greater than 0 millivolt (mV) to 70 millivolts, and the negative voltage based on the resting membrane potential comprises from less than 0 millivolt to −70 millivolts.

In some embodiments, a maximum current limit of the electrical pulse comprises from −1 milliampere (mA) and 1 milliampere.

In some embodiments, the electrical pulse comprises a single-phasic electrical pulse or a biphasic electrical pulse. The single-phasic electrical pulse comprising: the positive power or the negative power based on the extracellular membrane potential, or the positive voltage or the negative voltage based on the resting membrane potential. The biphasic electrical pulse comprising: the positive power and the negative power based on the extracellular membrane potential, or the positive voltage and the negative voltage based on the resting membrane potential.

In some embodiments, the biphasic electrical pulse comprises a biphasic asymmetric electrical pulse, the delivery frequency of the electrical pulse further comprises at least one pulse duration and at least one pulse dead time, and the at least one pulse duration is different from the at least one pulse dead time (also called interphase gap).

In some embodiments, the at least one pulse duration is from 2 seconds to 60 seconds, and the at least one pulse dead time is from 15 seconds to 300 seconds.

In some embodiments, a number of the at least one pulse duration is two pulse durations, a number of the at least one pulse dead time is two pulse dead times, and the pulse durations and the pulse dead times are sequentially arranged at intervals with each other.

In some embodiments, the biphasic electrical pulse further comprises: the positive power first and then the negative power, or the negative power first and then the positive power; or the positive voltage first and then the negative voltage, or the negative voltage first and then the positive voltage.

In some embodiments, the delivery frequency of the electrical pulse further comprises at least one pulse duration and at least one pulse dead time, wherein the at least one pulse duration is from 2 seconds to 60 seconds, and the at least one pulse dead time is from 15 seconds to 300 seconds.

In some embodiments, a number of the at least one pulse duration is two pulse durations, a number of the at least one pulse dead time is two pulse dead times, and the pulse durations and the pulse dead times sequentially arranged at intervals with each other.

In some embodiments, a number of the electrical pulse is from 1 to 30.

In some embodiments, the positive voltage based on the resting membrane potential or the positive power based on the extracellular membrane potential causes the plurality of cells in the target area to depolarize, wherein the negative voltage based on the resting membrane potential or the negative power based on the extracellular membrane potential causes the plurality of cells in the target area to hyperpolarize.

In some embodiments, a waveform of the electrical pulse comprises square, trapezoidal, peak, flat peak, or a combination thereof.

In some embodiments, pulse stimulator further comprises a flexible catheter and an electrode assembly. A proximal end of the flexible catheter connected to the generator. The electrode assembly comprising the at least one electrode disposed at a distal end of the flexible catheter, and electrically connected to the generator.

In some embodiments, a number of the at least one electrode is a plurality of electrodes, the plurality of electrodes comprise bipolar electrodes or tripolar electrodes.

In some embodiments, the generator comprises a power source unit and a control unit. The power source unit comprises a driver circuit block configured to generate, pause, or stop a signal of power, voltage, or current; and a power module is electrically connected to the driver circuit block, and is configured to control and output the signal to the electrode assembly. The control unit electrically connected to the driver circuit block, and configured to control generation and stop of the signal in the driver circuit block.

The present disclosure also provides a method for cell ablation, comprising: providing at least one electrode, the at least one electrode disposed on a plurality of cells in a target area; transmitting an electrical pulse to the at least one electrode; and ablating the plurality of cells in the target area, wherein the electrical pulse is configured to non-thermal ablate the plurality of cells in the target area, wherein the electrical pulse comprises: a positive voltage, a negative voltage, or a combination thereof based on a resting membrane potential; or a positive power, a negative power, or a combination thereof based on an extracellular membrane potential being 0 volt, and wherein a delivery frequency of the electrical pulse comprises from 0.001 Hz to less than 1 Hz.

In some embodiments, the step of ablating the plurality of cells in the target area comprises: inducing calcium ions to flow into the cells by the electrical pulse and to cause calcium ion permeability differences, thereby ablating the plurality of cells in the target area.

In some embodiments, an ohmic heat generated during the step of ablating the plurality of cells in the target area is less than 7.5×10⁻² joules.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion. The disclosure can be more fully understood by reading the following detailed description of the embodiment, with reference made to the accompanying drawings as follows:

FIG. 1 is a schematic view of a pulse stimulator according to some embodiments of the present disclosure.

FIG. 2 is a schematic view of a survival rate of MeT-5A cells under different biphasic electrical pulse times according to some embodiments of the present disclosure. **: p<0.01.

FIG. 3 is a schematic view of a survival rate of ES-2 cells under different biphasic electrical pulse times according to some embodiments of the present disclosure. **: p<0.01.

### DETAILED DESCRIPTION

The following disclosure provides detailed description of many different embodiments, or examples, for implementing different features of the provided subject matter. These are, of course, merely examples and are not intended to limit the invention but to illustrate it. In addition, various embodiments disclosed below may combine or substitute one embodiment with another, and may have additional embodiments in addition to those described below in a beneficial way without further description or explanation. In the following description, many specific details are set forth to provide a more thorough understanding of the present disclosure. It will be apparent, however, to those skilled in the art, that the present disclosure may be practiced without these specific details.

Further, spatially relative terms, such as “beneath,” “over” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, or “includes” and/or “including” or “has” and/or “having” when used in this specification, specify the presence of stated features, regions, integers, steps, operations, elements, and/or components, but do not preclude the presence or addition of one or more other features, regions, integers, steps, operations, elements, components, and/or groups thereof.

Further, when a number or a range of numbers is described with “about,” “approximate,” and the like, the term is intended to encompass numbers that are within a reasonable range considering variations that inherently arise during manufacturing as understood by one of ordinary skill in the art. For example, the number or range of numbers encompasses a reasonable range including the number described, such as within +/−10% of the number described, based on known manufacturing tolerances associated with manufacturing a feature having a characteristic associated with the number. This repetition is for the purpose of simplicity and clarity and does not in itself dictate a relationship between the various embodiments and/or configurations discussed.

Thermal ablation methods include radiofrequency ablation (RFA), microwave ablation (MWA), cryoablation (CRYO) and high-intensity focused ultrasound (HIFU); while non-thermal ablation methods include but are not limited to irreversible electroporation (IRE). The present disclosure also belongs to a non-thermal ablation method, the mechanism of which is to induce programmed cell death of tumor cells by opening ion channels to cause differences in calcium ion permeability, and it will not cause reversible or irreversible perforation in the cell membrane.

The present disclosure provides a pulse stimulator for cell ablation, including: a generator configured to deliver an electrical pulse to at least one electrode, in which the electrical pulse comprises a positive voltage, a negative voltage, or a combination thereof based on a resting membrane potential, and a delivery frequency of the electrical pulse comprises from 0.001 Hz to less than 1 Hz; wherein the electrical pulse is configured to non-thermal ablate a plurality of cells in a target area.

As used herein, “resting membrane potential” refers to the potential difference between the inner and outer sides of the cell membrane when the cell is in a resting state. The base point (0 V) is usually the potential outside the cell membrane. Depending on the species and cell type, the resting membrane potential can be from −20 mV to −200 mV. For example, when the resting membrane potential is −70 mV, the positive voltage based on the resting membrane potential can be understood as a voltage upward based on −70 mV, such as −60 mV, −30 mV, 0 mV, 30 mV, etc.; conversely, the negative voltage based on the resting membrane potential can be understood as a voltage downward based on −70 mV, such as −80 mV, −90 mV, etc. In the following examples, the resting membrane potential of the cell is used as the basic potential to provide a constant potential (+60 mV and −60 mV), which means, for example, if the resting membrane potential is −70 mV, +60 mV is given to make it −10 mV, and vice versa.

As used herein, “extracellular membrane potential” refers to that the extracellular membrane potential is 0 V.

In some examples, the positive power based on the extracellular membrane potential comprises from greater than 0 μW to 100 μW, and the negative power based on the extracellular membrane potential comprises from less than 0 μW to −100 μW. In some examples, the positive power of the electrical pulse includes, but is not limited to 10 W, 20 W, 30 μW, 40 μW, 50 μW, 60 μW, 70 μW, 80 μW, 90 μW, 100 μW, or any value between any two of these values. In some examples, the negative power of the electrical pulse includes, but is not limited to −10 μW, −20 μW, −30 μW, −40 μW, −50 W, −60 μW, −70 μW, −80 μW, −90 μW, −100 μW, or any value between any two of these values. In some examples, the power of the biphasic electrical pulse is from +10 μW to ±100 μW, for example, ±10 μW, ±20 μW, ±30 μW, ±40 μW, ±50 μW, ±60 μW, ±70 μW, ±80 μW, ±90 μW, ±100 μW, or any value between any two of these values. In some examples, the power of the biphasic electrical pulse can be positive or negative power with the same or different values.

In some examples, the positive voltage based on the resting membrane potential comprises from greater than 0 mV to 70 mV, and the negative voltage based on the resting membrane potential comprises from less than 0 mV to −70 mV. In some examples, electrical pulse that the positive voltage based on the resting membrane potential includes, bit is not limited to 10 mV, 20 mV, 30 mV, 40 mV, 50 mV, 60 mV, 70 mV, or any value between any two of these values. In some examples, electrical pulse that the negative voltage based on the resting membrane potential includes, but is not limited to −10 mV, −20 mV, −30 mV, −40 mV, −50 mV, −60 mV, −70 mV, or any value between any two of these values. In some examples, the voltage of the biphasic electrical pulse is from #10 mV to ±70 mV, for example, ±10 mV, ±20 mV, ±30 mV, ±40 mV, ±50 mV, ±60 mV, ±70 mV, or any value between any two of these values. In some examples, the voltage of the biphasic electrical pulse can be positive or negative voltage with the same or different values.

In some examples, a maximum current limit of the electrical pulse includes from −1 milliampere (mA) to 1 mA. When the current generated by the electrical pulse does not exceed the human body's perception current (1 mA), the disclosure has high safety.

In some examples, the electrical pulse includes: a single-phasic electrical pulse or a biphasic electrical pulse. The single-phasic electrical pulse is: the positive power or the negative power based on the extracellular membrane potential, or the positive voltage or the negative voltage based on resting membrane potential. The biphasic electrical pulse includes: the positive power or the negative power based on the extracellular membrane potential, or the positive voltage or the negative voltage based on the resting membrane potential.

In some examples, the biphasic electrical pulse includes biphasic asymmetric electrical pulse, a delivery frequency of the electrical pulse further includes at least one pulse duration and at least one pulse dead time, the at least one pulse duration and the at least one pulse dead time are not the same.

In some examples, the at least one pulse duration is from 2 seconds to 60 seconds, and the at least one pulse dead time is from 15 seconds to 300 seconds.

In some examples, a number of the at least one pulse duration is two pulse durations, a number of the at least one pulse dead time is two pulse dead times, and the pulse durations and the pulse dead times are sequentially arranged at intervals with each other.

In some examples, the pulse durations are selected from the group consisting of any two of times from 2 seconds to 60 second, and the pulse dead times are selected from the group consisting of any two of times from 15 seconds to 300 second.

In some examples, the biphasic electrical pulse further includes: the positive power first and then the negative power, or the negative power first and then the positive power; or the positive voltage first and then the negative voltage, or the negative voltage first and then the positive voltage.

In some examples, the delivery frequency includes from 0.001 Hz to less than 1 Hz, for example, 1/62 Hz, 1/92 Hz, 1/662 Hz, 1/720 Hz, or any value between any two of these values.

In some examples, a number of the electrical pulse is from 1 pulse to 30 pulses, for example, 2 pulses, 3 pulses, 4 pulses, 5 pulses, 6 pulses, 7 pulses, 8 pulses, 9 pulses, 10 pulses, 11 pulses, 12 pulses, 13 pulses, 14 pulses, 15 pulses, 16 pulses, 17 pulses, 18 pulses, 19 pulses, 20 pulses, 21 pulses, 22 pulses, 23 pulses, 24 pulses, 25 pulses, 26 pulses, 27 pulses, 28 pulses, 29 pulses, or 30 pulses.

In some examples, the positive voltage based on the resting membrane potential or the positive power based on the extracellular membrane potential causes the plurality of cells in the target area to depolarize, wherein the negative voltage based on the resting membrane potential or the negative power based on the extracellular membrane potential causes the plurality of cells in the target area to hyperpolarize.

In some examples, a waveform of the electrical pulse includes square, trapezoidal, peak, flat peak, or a combination thereof.

As used herein, “depolarize” refers to that when the cell membrane potential rises steeply from the resting membrane potential to a positive potential level, sodium, calcium and other ions with positively charge enter the cell membrane and increase the membrane potential.

As used herein, “hyperpolarize” refers to that the cells are directly produced a larger membrane potential difference by using external potential or power control, that is, the potential is lower than the resting membrane potential of the cells.

In some examples, the target area includes tumor tissue, or a mixture of tumor tissue and normal tissue.

The disclosed device provides a non-thermal ablation method that can destroy or eliminate tumors without high electric fields and high frequencies, and has better performance in inducing cell death and thermal control. A number of examples are provided herein to elaborate the pulse stimulator for cell ablation and method thereof of the instant disclosure. However, the examples are for demonstration purpose alone, and the instant disclosure is not limited thereto. The scope of protection of the present disclosure shall be determined by the scope of the claims attached hereto.

### EXAMPLE

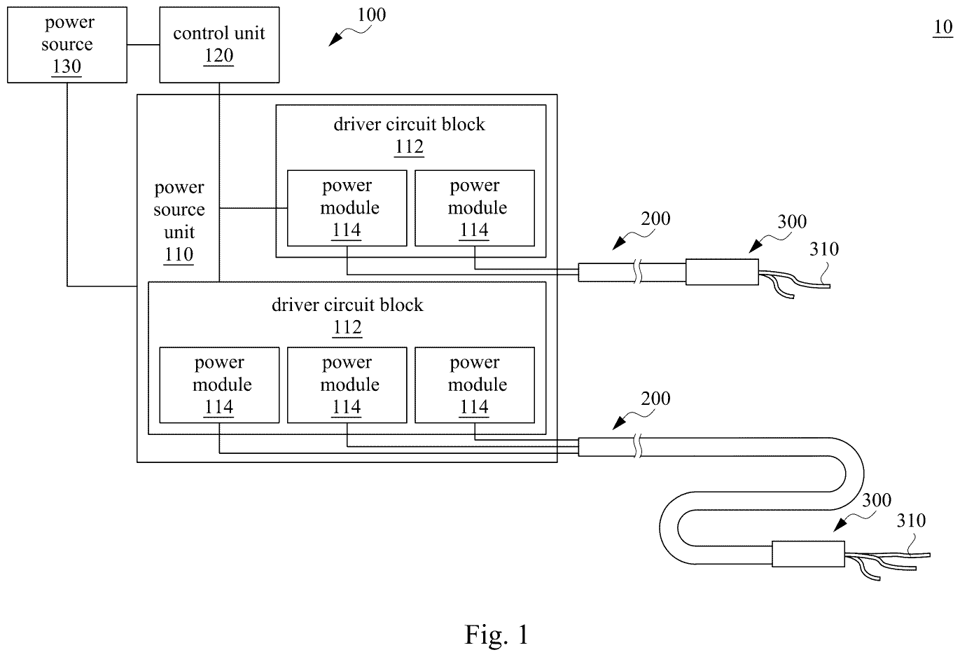

Please refer to FIG. 1, FIG. 1 is a schematic view of a pulse stimulator according to some embodiments of the present disclosure. The present disclosure provides a pulse stimulator 10 for cell ablation, including: a generator 100, a flexible catheter 200, and an electrode assembly 300.

The generator 100 is configured to deliver an electrical pulse to the electrode assembly 300, in which the electrical pulse is a positive voltage, a negative voltage, or a combination thereof based on a resting membrane potential, or is a positive power, a negative power, or a combination thereof based on an extracellular membrane potential being 0 V. A delivery frequency of the electrical pulse is from 0.001 Hz to less than 1 Hz. The electrical pulse is configured to non-thermal ablate a plurality of cells in a target area. In some examples, the generator 100 can continuously change the electrical pulse to power the electrode assembly 300.

Specificity, the generator 100 includes a power source unit 110, a control unit 120, and a power source 130. The power source unit 110 includes a driver circuit block 112 and a power module 114. The driver circuit block 112 is configured to generate, pause, or stop a signal of power, voltage, or current. The power module 114 is electrically connected to the driver circuit block 112, and is configured to control and output the signal to the electrode assembly 300. The power module 114 is selectively controlled by the driver circuit block 112 to continuously change the power level associated with the signal. In some examples, the power source unit 110 further includes a filter and an electrical isolation block. The control unit 120 is electrically connected to the driver circuit block 112 in the power source unit 110, and is configured to control the generation and stop of the signal in the driver circuit block 112.

The power source 130 is electrically connected to the power source unit 110 and the control unit 120 to provide the required source of electricity. In some examples, the power source 130 provides alternating current (AC) power. For example, the power source 130 can be battery powered or connected to an AC grid capable of producing 110 volts or 240 volts.

The flexible catheter 200 includes a proximal end and a distal end opposite to the proximal end, the proximal end of the flexible catheter is connected to the generator 100. In some examples, a material of the flexible catheter 200 is biocompatible material and is flexible. In some examples, the flexible catheter 200 has a wire inside, and both ends of the wire are electrically connected to the generator 100 and the electrode assembly 300, respectively. Specifically, the flexible catheter 200 penetrates into the blood vessel to reach the target area, and the target area includes, but is not limited to, organs, tissues or body walls. In other examples, the flexible catheter 200 can also penetrate into the respiratory tract, birth canal, skin, or mucosal openings, and directly contact the skin or mucosa.

The electrode assembly 300 includes at least one electrode 310, at least one electrode 310 disposed at the distal end of the flexible catheter 200, and the electrode assembly 300 is electrically connected to the generator 100. In some examples, a number of the at least one electrode 310 is a plurality of electrodes 310. In some examples, the plurality of electrodes 310 include, but are not limited to bipolar electrodes or tripolar electrodes. The electrodes 310 are powered by the single power source unit 110 through an electrical signal to deliver power for treating or ablating tissue. In some examples, a material of the electrode 310 includes, but is not limited to, gold, platinum, silver/silver chloride, carbon electrode, indium tin oxide (ITO), or a combination thereof.

In some examples, power source unit 110 includes at least one driver circuit block 112, the at least one driver circuit block 112 has at least one power module 114, for example a plurality of power modules 114. Each one of the plurality of power modules 114 can provide a positive power or a negative power, or a positive voltage or a negative voltage in a single time period. The wires extending from the plurality of power modules 114 can be connected together to an electrode assembly 300. One electrode assembly 300 includes at least one electrode 310, for example a plurality of electrodes 310. The plurality of power modules 114 respectively correspond to the plurality of electrode 310 (for example, WE+CE (working electrode and counter electrode) or WE+CE+RE (reference electrode)).

In other examples, the power source unit 110 includes a driver circuit block 112 and a power module 114. The at least one electrode 310 is monopolar electrode 310, and the monopolar electrode 310 is electrically connected to one power module 114 of the power source unit 110.

In other examples, the power source unit 110 includes one driver circuit block 112 and two power modules 114. The at least one electrode 310 is bipolar electrodes 310, and the bipolar electrodes 310 are electrically connected to the two power modules 114 respectively, and further electrically connected to the power source unit 110. The bipolar electrodes 310 respectively are working electrode (WE) and counter electrode (CE). In the two power modules 114, one provides positive power, the other one provides negative power and provides alternatively; or one provide positive voltage, the other one provide negative voltage and provides alternatively, so the electrical pulse is formed as biphasic electrical pulse.

In other examples, the power source unit 110 includes one driver circuit block 112 and three power modules 114. The at least one electrode 310 is tripolar electrodes 310, and the tripolar electrodes 310 are electrically connected to the three power modules 114 of the power source unit 110 respectively. The bipolar electrodes 310 respectively are WE, CE, and RE. In the three power modules 114, one provides positive power, another provides negative power, the other provides zero power, so the electrical pulse us formed as biphasic electrical pulse. In detail, the differences in the power modules 114 are that the output powers are different. That is, different power outputs have their own limitations or output ranges, and the power conditions provided by the three power modules 114 can be adjusted according to demand. In some examples, the first power module is responsible for power outputs above 100 μW, the second power module is responsible for power outputs between 25 and 100 μW, and the third power module is responsible for power outputs below 25 μW.

In some examples, power source unit 110 includes two driver circuit blocks 112, one of the driver circuit blocks 112 has two power modules 114, the other one of the driver circuit blocks 112 has three power modules 114. It should be understood that the group states included in the power source unit 110 can be adjusted according to needs, and the above is only an example and is not intended to be limiting.

Although a series of operations or steps are used below to describe the method disclosed herein, an order of these operations or steps should not be construed as a limitation to the present invention. For example, some operations or steps may be performed in a different order and/or other steps may be performed at the same time. In addition, all shown operations, steps and/or features are not required to be executed to implement an embodiment of the present invention. In addition, each operation or step described herein may include a plurality of sub-steps or actions.

### Example 1

1×10⁵ ES-2 cells (ovarian cancer cells) were evenly cultured on the surface of the ITO electrode and placed in McCoy's 5A culture medium. After two days of culture, the resting membrane potential of the cells was used as the basic potential, constant potential (+60 mV and −60 mV), and different pulse dead times (15 seconds, 120 seconds and 300 seconds) and pulse durations (2 seconds, 30 seconds and 60 seconds) stimulation were provided. After the stimulation was completed, a membrane potential difference between the final potential and potential before the stimulation was calculated as a reference for future electrical stimulation.

TABLE 1

| Constant | | | | | | | | |
|---|---|---|---|---|---|---|---|---|
| potential | | | | | | Mean | | |
| (relative to | | Pulse | Data | Membrane | | intracellular | | |
| resting | Pulse | dead | collection | potential | Mean | calcium | Mean | |
| membrane | duration | time | time | difference | survival | fluorescence | current | Joule |
| potential) | (secs) | (secs) | (secs) | (mV) | rate | (a.u.) | (mA) | (J) |
| 0 mV | — | — | — | <0.01000 | 1.00000 | 21.72131 | — | — |
| +60 mV | 2 | — | 15 secs | 0.01916 | 0.95235 | 16.64001 | 2.17870 | 0.00026 |
| | 30 | — | after | 0.03002 | 0.90663 | — | 0.18250 | 0.00033 |
| | 60 | — | single | 0.03446 | 0.83690 | 62.64606 | 0.13036 | 0.00047 |
| | 60 | 15 | pulse | 0.01856 | | | | |
| | | 120 | | 0.01772 | | | | |
| | | 300 | | 0.02125 | | | | |
| −60 mV | 2 | — | | −0.02148 | 1.09337 | 13.33211 | −3.40430 | 0.00041 |
| | 30 | — | | −0.02348 | 1.10241 | — | −0.20720 | 0.00037 |
| | 60 | — | | −0.03385 | 0.78967 | 21.86951 | −0.09824 | 0.00035 |
| | 2 | 15 | | −0.02212 | | | | |
| | | 120 | | −0.02698 | | | | |
| | | 300 | | −0.01706 | | | | |

The results were shown in Table 1 above. Since there was no significant difference in pulse dead time whether it is 15 seconds, 120 seconds, or 300 seconds after the +60 mV pulse, 15 seconds was used as the interval time of the biphasic electrical pulse. Then, when the pulse duration was 60 seconds, ES-2 cells had the lowest survival rate and the maximum intracellular calcium ion influx.

The experimental results showed that a fixed 60 mV can obtain a maximum current range of 8.0 mA to 10 mA and an average current range of 0.09 mA to 3.4 mA. The cell resistance was calculated by V=IR to be about 6Ω to 666.7Ω. Since the current value that the human body can perceive was 1 mA, this was used as the basis for current limitation. Therefore, subsequent experiments continued to stimulate cells with a fixed power, and the cell membrane potential range can be obtained to be about 6 mV to 666.7 mV.

### Example 2

The experiment was divided into the following four groups:
MeT-5A control group: 1×10⁵ MeT-5A cells (normal epithelial cells) were evenly cultured on the surface of the ITO electrode and placed in Medium 199. After two days of culture, the survival rate was calculated.
ES-2 control group: 1×10⁵ ES-2 cells (ovarian cancer cells) were evenly cultured on the surface of the ITO electrode and placed in McCoy's 5A culture medium. After two days of culture, the survival rate and intracellular calcium fluorescence value were calculated.
MeT-5A experimental group, ES-2 experimental group: The culture conditions were the same as those of the MeT-5A and ES-2 control groups. The difference was that after two days of culture, the cells were stimulated with a fixed power (+15 μW/−15 μW or +30 μW/−30 μW) and a maximum current range (+1 mA) at the extracellular membrane potential (OV) of the cells for different times (12 times, 15 times, 18 times, 24 times) of biphasic asymmetric pulse cycles. For example, +15 μW for 60 seconds, rest for 15 seconds, −15 μW for 2 seconds, and rest for 15 seconds constituted one biphasic asymmetric pulse cycle. After the stimulation, the cell survival rate, intracellular calcium fluorescence value, mean transient current, current average value, and Joule heat were calculated.

TABLE 2

| | Electrical pulse condition | | | | | | | | Current (mA) | | |
|---|---|---|---|---|---|---|---|---|---|---|---|
| | | | pulse | | | pulse | | | Mean | | Joule |
| | | pulse | dead | | pulse | dead | Pulse | Survival | transient | Mean | heat |
| Cell | Power | duration | time | Power | duration | time | times | rate | current | current | Joule |
| Line | (μW) | (secs) | (secs) | (μW) | (secs) | (secs) | Numbers | Mean | (mA) | (mA) | (J) |
| Control | — | — | — | — | — | — | — | 1.00000 | — | — | — |
| ES-2 | | | | | | | | | | | |
| ES-2 | +15 | 60 | 15 | −15 | 2 | 15 | 12 | 0.67993 | 0.72060 | 0.03990 | 0.01116 |
| | | | | | | | 15 | 0.58168 | 0.68780 | 0.03580 | 0.01395 |
| | | | | | | | 18 | 0.47573 | 0.59450 | 0.03580 | 0.01674 |
| | | | | | | | 24 | 0.41827 | 0.60774 | 0.11673 | 0.02232 |
| | +30 | 60 | 15 | −30 | 2 | 15 | 3 | 0.71719 | 0.22300 | 0.06610 | 0.00558 |
| | | | | | | | 12 | 0.37393 | 0.23120 | 0.04090 | 0.02232 |
| | | | | | | | 18 | 0.10826 | 0.17800 | 0.03680 | 0.03348 |
| | +45 | 60 | 15 | −45 | 2 | 15 | 12 | 0.33898 | 0.19830 | 0.04490 | 0.03348 |
| | +75 | 60 | 15 | −75 | 2 | 15 | 12 | 0.19608 | 0.33570 | 0.03280 | 0.05580 |
| | +100 | 60 | 15 | −100 | 2 | 15 | 12 | 0.20644 | 0.68270 | 0.07320 | 0.07440 |
| Control | — | — | — | — | — | — | — | 1.0000 | — | — | — |
| MeT-5A | | | | | | | | | | | |
| MeT-5A | +15 | 60 | 15 | −15 | 2 | 15 | 12 | 0.84199 | 0.50240 | 0.02650 | 0.01116 |
| | | | | | | | 15 | 0.75079 | 0.63630 | 0.03810 | 0.01395 |
| | | | | | | | 18 | 0.61738 | 0.63970 | 0.02510 | 0.01674 |
| | | | | | | | 24 | 0.56623 | 0.13223 | 0.00575 | 0.02232 |
| | +30 | 60 | 15 | −30 | 2 | 15 | 3 | 0.33617 | 0.22900 | 0.06740 | 0.00558 |
| | | | | | | | 12 | 0.08113 | 0.28700 | 0.04950 | 0.02232 |

*Each condition in the above groups was repeated 20 times, n = 3~20.

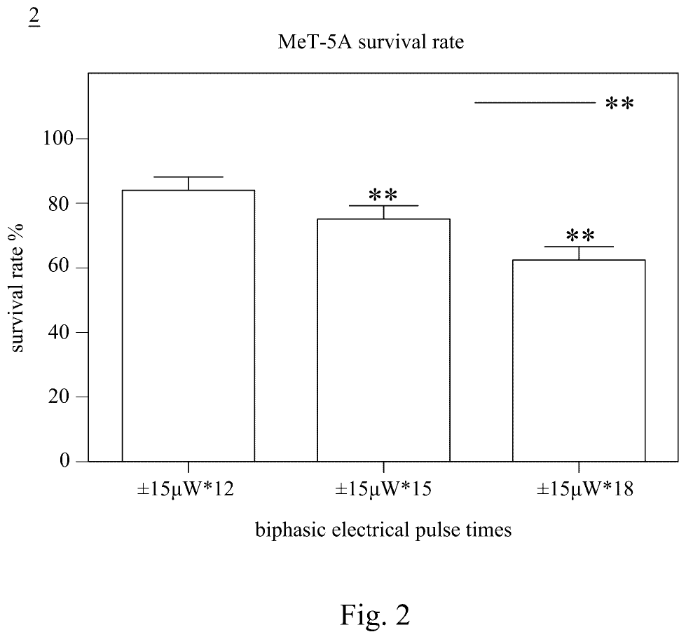

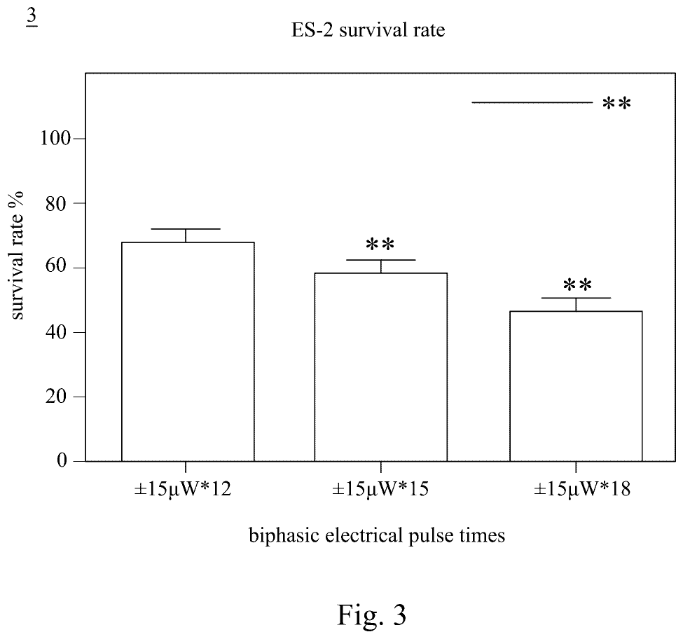

FIG. 2 is a schematic view 2 of a survival rate of MeT-5A cells under different biphasic electrical pulse times according to some embodiments of the present disclosure. FIG. 3 is a schematic view 3 of a survival rate of ES-2 cells under different biphasic electrical pulse times according to some embodiments of the present disclosure.

The results were shown in Table 2, FIG. 2 and FIG. 3. Compared with the ES-2 control group, the ES-2 experimental group significantly reduced the cell survival rate at 12, 15, 18 and 24 biphasic asymmetric pulse times; compared with the MeT-5A experimental group, the ES-2 experimental group also significantly reduced the cell survival rate. In addition, compared with the ES-2 control group (21.72 a.u.), the intracellular calcium fluorescence intensity (43.28 a.u.) of the ES-2 experimental group was significantly different, and the intracellular calcium concentration was positively correlated with the lethal effect, indicating that the present disclosure can affect the intracellular calcium ion homeostasis. Furthermore, the transient current did not exceed the human body perception current (1 mA), indicating that the present disclosure has high safety.

It is worth noting that when the number of pulse stimulations reaches 18 times, the survival rate of the ES-2 experimental group (ovarian cancer cells) was less than 50%, while the survival rate of the MeT-5A experimental group is greater than 50%. This showed that under the conditions of the same power and the same number of pulses, the survival rate of normal epithelial cells (MeT-5A) was significantly higher than that of ovarian cancer cells (ES-2). Therefore, the present disclosure had a certain protective effect (sub-lethal effects) on normal cells, and had a strong toxic effect (lethal effects) on cancer cells, indicating that this condition has a potential effect on mixed tissues (normal cells/tissues+cancer cells/tissues).

In addition, when the power of the ES-2 experimental group was increased, the survival rate continued to decrease, and the number of 50% lethal pulses could also be reduced, indicating that this condition can be used to focus on pure tumor tissue ablation, and the operation time can be further shortened. As for the MeT-5A experimental group, when the power intensity reached 30 μW, it had a significant toxic effect on normal cells. This showed that this condition was suitable for pure tumor tissue. When normal tissue and tumor tissue are mixed, the applicable power intensity is less than 30 μW, including but not limited to 15 μW.

### Example 3

The experiment was divided into two groups. 1×10⁵ ES-2 cells were evenly cultured on the surface of the ITO electrode and placed in McCoy's 5A culture medium. After two days of culture, calcium ion chelator (ethylene glycol tetraacetic acid (EGTA)) was added to both groups to completely remove extracellular calcium ions. One group (control group) was not stimulated by electrical pulse, and the other group (experimental group) was stimulated by 18 biphasic asymmetric pulse cycles with fixed power (+15 μW/−15 μW) and maximum current limit range (±1 mA). After the stimulation was completed, the cell survival rate, intracellular calcium fluorescence value, mean transient current, current average value, and Joule heat were calculated.

TABLE 3

| | Electrical pulse condition | | | | | | | | intracellular | | | |
|---|---|---|---|---|---|---|---|---|---|---|---|---|
| Cell | | | pulse | | | pulse | | | calcium | Current (mA) | | Joule |
| line | | pulse | dead | | pulse | dead | Pulse | Survival | fluorescence | Mean | | heat |
| adding | Power | duration | time | Power | duration | time | times | rate | (a.u.) | transient | Mean | Joule |
| EGTA | (μW) | (secs) | (secs) | (μW) | (secs) | (secs) | Numbers | Mean | Mean | current | current | (J) |
| ES-2 + | — | — | — | — | — | — | — | 0.917831 | 21.063413 | — | — | — |
| EGTA | | | | | | | | | | | | |
| ES-2 + | +15 | 60 | 15 | −15 | 2 | 15 | 18 | 0.790077 | 24.214893 | 0.7869 | 0.081000 | 0.016740 |
| EGTA | | | | | | | | | | | | |

*Each condition in each group above was repeated at least 3 times, n > 3.

The results were shown in Table 3 above. The survival rate results showed that the survival rate of cancer cells in the experimental group was significantly increased from 47.57% (the group with the same conditions in Table 2) to 79.00%, and the intracellular calcium concentration was the same as that of the control group. This indicates that the present disclosure can induce calcium ions to flow into cells, and the cause of cell death is related to calcium ion homeostasis.

Therefore, the cause of ablation caused by the present disclosure is different from the conventional one. The IRE method directly causes irreversible cell membrane damage, while the reversible electroporation method causes metabolic incapacity of abnormal tissues or cells through the combination of temperature control or additional delivery of drugs, genes or ions into cells. The present disclosure has been experimentally confirmed (compared with cancer cells without pulse stimulation) that after pulse stimulation, tumor cells can show calcium ion permeability differences, achieving the goal of inducing programmed cell death of tumor cells. It can be seen that the membrane damage mechanism caused by electroporation or ablation in the aforementioned is different from the mechanism of calcium ion permeability difference caused by the present disclosure. When the electrical pulse provides a voltage higher than the resting membrane potential or a positive power higher than the extracellular membrane potential, calcium ions are induced to flow into the cell membrane. When the electrical pulse provides a voltage lower than the resting membrane potential or a positive power lower than the extracellular membrane potential, all or part of the calcium ions in the cell are induced to flow into the mitochondria to induce programmed cell death in tumor cells.

While the disclosure has been described by way of example(s) and in terms of the preferred embodiment(s), it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A pulse stimulator for cell ablation, comprising:
a generator configured to deliver an electrical pulse to at least one electrode, the electrical pulse comprising:
a positive voltage, a negative voltage, or a combination thereof based on a resting membrane potential; or
a positive power, a negative power, or a combination thereof based on an extracellular membrane potential being 0 volt,
wherein a delivery frequency of the electrical pulse comprises from 0.001 Hz to less than 1 Hz, and
wherein the electrical pulse is configured to non-thermal ablate a plurality of cells in a target area.

2. The pulse stimulator of claim 1, wherein the positive power based on the extracellular membrane potential comprises from greater than 0 microwatt to 100 microwatts, and the negative power based on the extracellular membrane potential comprises from less than 0 microwatt to −100 microwatts.

3. The pulse stimulator of claim 1, wherein the positive voltage based on the resting membrane potential comprises from greater than 0 millivolt to 70 millivolts, and the negative voltage based on the resting membrane potential comprises from less than 0 millivolt to −70 millivolts.

4. The pulse stimulator of claim 1, wherein a maximum current limit of the electrical pulse comprises from −1 milliampere and 1 milliampere.

5. The pulse stimulator of claim 1, wherein the electrical pulse comprises:
a single-phasic electrical pulse comprising: the positive power or the negative power based on the extracellular membrane potential, or the positive voltage or the negative voltage based on the resting membrane potential; or
a biphasic electrical pulse comprising: the positive power and the negative power based on the extracellular membrane potential, or the positive voltage and the negative voltage based on the resting membrane potential.

6. The pulse stimulator of claim 5, wherein the biphasic electrical pulse comprises a biphasic asymmetric electrical pulse, the delivery frequency of the electrical pulse further comprises at least one pulse duration and at least one pulse dead time, and the at least one pulse duration is different from the at least one pulse dead time.

7. The pulse stimulator of claim 6, wherein the at least one pulse duration is from 2 seconds to 60 seconds, and the at least one pulse dead time is from 15 seconds to 300 seconds.

8. The pulse stimulator of claim 7, wherein a number of the at least one pulse duration is two pulse durations, a number of the at least one pulse dead time is two pulse dead times, and the pulse durations and the pulse dead times are sequentially arranged at intervals with each other.

9. The pulse stimulator of claim 5, wherein the biphasic electrical pulse further comprises:
the positive power first and then the negative power, or the negative power first and then the positive power; or
the positive voltage first and then the negative voltage, or the negative voltage first and then the positive voltage.

10. The pulse stimulator of claim 1, wherein the delivery frequency of the electrical pulse further comprises at least one pulse duration and at least one pulse dead time, wherein the at least one pulse duration is from 2 seconds to 60 seconds, and the at least one pulse dead time is from 15 seconds to 300 seconds.

11. The pulse stimulator of claim 10, wherein a number of the at least one pulse duration is two pulse durations, a number of the at least one pulse dead time is two pulse dead times, and the pulse durations and the pulse dead times sequentially arranged at intervals with each other.

12. The pulse stimulator of claim 1, wherein a number of the electrical pulse is from 1 to 30.

13. The pulse stimulator of claim 1, wherein the positive voltage based on the resting membrane potential or the positive power based on the extracellular membrane potential causes the plurality of cells in the target area to depolarize, wherein the negative voltage based on the resting membrane potential or the negative power based on the extracellular membrane potential causes the plurality of cells in the target area to hyperpolarize.

14. The pulse stimulator of claim 1, wherein a waveform of the electrical pulse comprises square, trapezoidal, peak, flat peak, or a combination thereof.

15. The pulse stimulator of claim 1, further comprising:
a flexible catheter, a proximal end of the flexible catheter connected to the generator; and
an electrode assembly comprising the at least one electrode disposed at a distal end of the flexible catheter, and electrically connected to the generator.

16. The pulse stimulator of claim 15, wherein a number of the at least one electrode is a plurality of electrodes, the plurality of electrodes comprise bipolar electrodes or tripolar electrodes.

17. The pulse stimulator of claim 15, wherein the generator comprises:
a power source unit comprising:
a driver circuit block configured to generate, pause, or stop a signal of power, voltage, or current; and
a power module is electrically connected to the driver circuit block, and is configured to control and output the signal to the electrode assembly; and
a control unit electrically connected to the driver circuit block, and configured to control generation and stop of the signal in the driver circuit block.

18. A method for cell ablation, comprising:
providing at least one electrode, the at least one electrode disposed on a plurality of cells in a target area;
transmitting an electrical pulse to the at least one electrode; and
ablating the plurality of cells in the target area, wherein the electrical pulse is configured to non-thermal ablate the plurality of cells in the target area,
wherein the electrical pulse comprises:
a positive voltage, a negative voltage, or a combination thereof based on a resting membrane potential; or
a positive power, a negative power, or a combination thereof based on an extracellular membrane potential being 0 volt, and
wherein a delivery frequency of the electrical pulse comprises from 0.001 Hz to less than 1 Hz.

19. The method of claim 18, wherein the step of ablating the plurality of cells in the target area comprises:
inducing calcium ions to flow into the cells by the electrical pulse and to cause calcium ion permeability differences, thereby ablating the plurality of cells in the target area.

20. The method of claim 18, wherein an ohmic heat generated during the step of ablating the plurality of cells in the target area is less than 7.5×10⁻² joules.
